(19) United States
(12) Patent Application Publication
(10) Pub. No.: US 2020/0273537 A1
(43) Pub. Date: Aug. 27, 2020

(54) High Throughput Patient Genomic Sequencing and Clinical Reporting Systems

(71) Applicant: Nantomics, Culver City, CA (US)

(72) Inventors: Patrick Soon-Shiong, Culver City, CA (US); Shahrooz Rabizadeh, Agoura Hills, CA (US); Stephen Charles Benz, Culver City, CA (US); John Zachary Sanborn, Culver City, CA (US); Charles Joseph Vaske, Culver City, CA (US)

(21) Appl. No.: 16/859,826

(22) Filed: Apr. 27, 2020

Related U.S. Application Data
(63) Continuation of application No. 15/015,048, filed on Feb. 3, 2016.
(60) Provisional application No. 62/162,530, filed on May 15, 2015.
(60) Provisional application No. 62/112,026, filed on Feb. 4, 2015.
(60) Provisional application No. 62/111,607, filed on Feb. 3, 2015.

(51) Int. Cl. G16B 20/00; G16B 50/00

(52) U.S. Cl. CPC G16B 20/00; G16B 50/00

(57) ABSTRACT

Contemplated panomic systems and methods significantly improve accuracy of genetic testing by taking into account matched normal data and expression levels of various genes in diseased tissue. Analysis and physician guidance is further improved by combining so identified clinically relevant changes with pathway analysis to thereby allow for classification of a tumor and/or identification of potentially druggable targets within affected pathways.

This application is a continuation of U.S. patent application Ser. No. 15/015,048 filed 3 Feb. 2016, which in turn claims the benefit of priority to U.S. provisional application 62/111607, which was filed 3 Feb. 2015, 62/112026, which was filed 4 Feb. 2015, and 62/162530, which was filed 15 May 2015, all of which are incorporated by reference herein.

### FIELD OF THE INVENTION

The field of the invention is systems and methods for omics-based data analysis in patient evaluation and treatment outcome prediction.

### BACKGROUND OF THE INVENTION

The background description includes information that may be useful in understanding the present invention. It is not an admission that any of the information provided herein is prior art or relevant to the presently claimed invention, or that any publication specifically or implicitly referenced is prior art.

All publications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Most clinicians today are challenged by a deluge of new insights from rapidly advancing science with which it becomes increasingly difficult to keep pace. With respect to new treatment options, nearly 800 drugs are in development to target specific proteins involved in driving tumor growth. However, efficacy and proper patient selection criteria for these new drugs are not fully understood. With respect to molecular diagnostics, tests identifying gene mutations that may be relevant in signaling pathways have revealed hundreds of ‘actionable’ genes that may be subject to pharmaceutical intervention. Unfortunately, such limited set of selected genes is almost entirely restricted to functionally characterized corresponding proteins, which is entirely unsuitable to provide a comprehensive overview of genetic interactions and compensatory mechanisms.

Moreover, genome sequencing data of a tumor per se may not necessarily provide the specific information that indicates that a mutation is in fact foundational to tumor development or growth. For example, the risks and disadvantages of analyzing DNA from tumor samples alone without matched normal samples (i.e., sample from non-diseased healthy tissue of the same patient) was recently highlighted by Jones et al. (Jones S, Anagnostou V, Lytle K, et al. Personalized genomic analyses for cancer mutation discovery and interpretation. Sci Transl Med. 2015; 283.) reporting that as many as one third of actionable changes identified by gene panels may be incorrectly classified as somatic (tumor) changes when they actually represent germline (non-tumor) alterations. Viewed from a different perspective, not every change in a genome represents a cause or contributing factor in the development and growth of a tumor. On that backdrop, when the complete set of more than 20,000 coding genes (whole exome) was studied, the false-positive rate rose to 65%.

In addition, the exclusive reliance on genotyping of tumor samples to drive treatment decisions is largely based on the assumption that identification of mutated DNA will routinely translate to an alteration in the underlying protein pathways that are targeted by the therapy. Unfortunately, in such conceptual model a genome sequence could be considered merely a map, but not the actual treatment territory. Consequently, even if a patient's whole genome were available, such information would not necessarily give clinically relevant insight into treatment. Viewed from a different perspective, a genome-only approach will be prone to substantial false positive rates and will therefore unnecessarily expose a patient to ineffective and often highly toxic treatment.

Thus, even though significant advances in omics research and clinical therapy have been made, all of almost all of them suffer from several drawbacks. Consequently, there is still a need to provide rapid and clinically meaningful data for treatment of cancer.

### SUMMARY OF THE INVENTION

The inventors have discovered that to accurately predict the downstream effects of gene alterations, patient- and tumor-specific DNA information is needed, along with orthogonal technologies such as determination of RNA transcription levels, RNA sequencing, proteomics, and analysis of cell signaling pathways. RNA sequencing advantageously confirms the presence of mutations identified by DNA sequencing, enables the quantitation of gene expression, and can be used to infer the expression of proteins within actionable signaling pathways. Cell signaling pathway-based modeling algorithms such as PARADIGM allow integration of DNA sequencing and RNA sequencing information with known signaling pathways, and thereby facilitate the identification of key altered regulatory networks.

In one aspect of the inventive subject matter, the inventors contemplate a method of calculating a treatment recommendation using omics information. Especially preferred methods include a step of obtaining, by an analysis engine, from an omics data base or sequencing facility (1) omic information of a patient, wherein the omic information is generated from genomic sequence information of a genomic sequence in a diseased and a matched normal sample, and (2) a transcription level for the genomic sequence in at least the diseased sample. In another step, the omic information and the transcription level for the genomic sequence in at least the diseased sample is used by the analysis engine in a pathway model to calculate a pathway activity of a pathway containing the genomic sequence, and in yet another step, the analysis engine identifies a druggable target based on the calculated pathway activity. The calculated pathway activity is then used to update or generate a patient record with a treatment recommendation.

While not limiting to the inventive subject matter, it is generally preferred that the omic information comprises a differential sequence object that further comprises the transcription level for the genomic sequence in at least the diseased sample, and/or that the pathway model comprises an ensemble of treatment models, a trained treatment outcome prediction model, a pathway expression model, a pathway recognition algorithm using data integration on genomic models (PARADIGM), or a drug response model.

It is still further contemplated that the omic information of the patient and the transcription level are coordinately provided (e.g., at least two sequence data strings provided in the same transmission) from a sequencing facility. In addition, it is preferred that contemplated methods will also include a step of obtaining RNA sequence information for the transcribed genomic sequence in at least the diseased sample. Most typically, the RNA transcription data are over-represented relative to the DNA information. For example, the omic information may be built from less than 20× reads (i.e., coverage of a single base by less than 20 reads), and wherein the transcription level is obtained from at least 150× reads (i.e., quantification of a single transcript by more than 150 reads).

Similarly, the inventors also contemplate a method of calculating a treatment recommendation using omics information that includes a step of obtaining, by an analysis engine, from an omics data base or sequencing facility (1) omic information of a patient, wherein the omic information is generated from genomic sequence information of a genomic sequence in a diseased and a matched normal sample, and (2) a transcription level for the genomic sequence in at least the diseased sample. In another step, the omic information and the transcription level for the genomic sequence in at least the diseased sample is used by the analysis engine in a pathway model to calculate a pathway activity of a pathway containing the genomic sequence, and in yet another step, the analysis engine classifies a tumor based on the calculated pathway activity, wherein the step of classifying is performed independent of tumor anatomy. The classification of the tumor is then used to update or generate a patient record with a treatment recommendation.

As noted above, it is generally contemplated that the omic information comprises a differential sequence object that may further comprise the transcription level for the genomic sequence in at least the diseased sample. Moreover, it is generally preferred that the pathway model may be an ensemble of treatment models, a trained treatment outcome prediction model, a pathway expression model, a pathway recognition algorithm using data integration on genomic models (PARADIGM), or a drug response model.

It is still further contemplated that the omic information of the patient and the transcription level are coordinately provided (e.g., at least two sequence data strings provided in the same transmission) from a sequencing facility. In addition, it is preferred that contemplated methods will also include a step of obtaining RNA sequence information for the transcribed genomic sequence in at least the diseased sample. Most typically, the RNA transcription data are over-representated relative to the DNA information. For example, the omic information may be built from less than 20× reads (i.e., coverage of a single base by less than 20 reads), and wherein the transcription level is obtained from at least 150× reads (i.e., quantification of a single transcript by more than 150 reads).

Additionally, it is contemplated that classification may be performed based on a known mechanism of action for a drug, or based on increased or decreased pathway usage. Moreover, classification may also include designating a tumor as having MSI when the ratio of breakpoints per gigabase is above a predetermined frequency.

Consequently, the inventors also contemplate a high throughput computer-based genomic analysis system that includes at least one multi-lane sequencing device configured to sequence at least one patient's normal tissue and diseased tissue in a common run, wherein the at least one sequencing device is further configured to generate a genome sequence, an exome sequence and an RNA sequence of the normal and disease tissues by sequencing a genome, an exome, and RNA of the tissues, and wherein the exome sequence and RNA sequence are enriched relative to the genome sequence by at least a factor of five. Such systems will further include a modeling computer system that comprises at least one processor, at least one memory, and a modeling and reporting engine executable on the at least one processor: according to software instructions store in the at least one memory and configured to: store the genome sequence, the exome sequence, and the RNA sequence of the normal tissue and the diseased tissue of at least one patient in the at least one memory, store at least one sequence-based treatment model in the at least one memory, wherein the at least one sequence-based treatment model is programmed to generate clinical report data as a function of sequence data, generate patient-specific clinical report data in the at least one memory by executing the at least one sequence-based treatment model on at least one of the genome sequence, the exome sequence and the RNA sequence of the at least one patient, generate a clinical report from the patient-specific clinical report data, and cause an output device to present the clinical report.

In further contemplated systems, the at least one multi-lane sequencing device is further configured to sequence at least eight patient's normal tissue and diseased in the common run, or wherein the at least one multi-lane sequencing device includes at least ten one multi-lane sequencing devices. Moreover, it is generally preferred that the genome sequence comprises less than 20× reads, the exome sequence comprises at least 150× reads, and that the RNA sequence comprises at least 150× reads. Thus, in most typical instances, exome sequence or the RNA sequence is enriched by at least a factor of 10 relative to the genome sequence.

Additionally, it is generally preferred that the at least one treatment model comprises an ensemble of treatment models, a trained treatment outcome prediction model, pathway expression model, a pathway recognition algorithm using data integration on genomic models (PARADIGM), or a drug response model, and/or that the at least one of the genome sequence, the exome sequence, and the RNA sequence is stored in the at least one memory according to a BAMBAM format.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWING

FIG. 1 is an exemplary comparative schematic of currently known gene panel analysis vis-à-vis in contrast to panomic analysis according to the inventive subject matter.

FIG. 2 illustrates exemplary comparative graphs depicting patient results for highly expressed mutations in melanoma, lung, breast, and uterine cancers in Panel A, and low expressed mutations in certain cancers in Panel B. Panel C exemplarily shows associations between RNA expression and DNA alterations for each gene in a 328-gene panel.

FIG. 3 illustrates exemplary graphs depicting re-arrangement of genomic DNA as seen by WGS in Panel A and associated survival curves in Panel B. Panel 3C illustrates exemplary MSI status predictions using panomic analysis.

FIG. 4 illustrates graphs (Panel A: heat map; Panel B: bar graph) depicting exemplary stratification of various cancers into certain categories by pathway features rather than anatomic features.

FIGS. 5 is an exemplary schematic illustrating potential points of failure using incomplete omics analysis.

FIG. 6 is an exemplary outcome of a treatment recommendation for a patient diagnosed having end-stage cervical cancer.

### DETAILED DESCRIPTION

The inventors have now discovered that the widely held assumption that DNA mutations always give rise to possible downstream protein targets is inaccurate. Indeed, and as is further shown in more detail below, analysis of DNA without confirmatory RNA expression (and RNA sequence information) may lead to false-positive, false-negative, and missed findings, potentially resulting in inappropriate treatment selection.

For example, the inventors identified such inaccuracies from a large scale 3,784 patient omic data set (DNA and RNA sequencing) covering 19 anatomical tumor types. The data set was processed to detect patient specific DNA variants (i.e., tumor versus matched normal; germline vs somatic) and RNA expression to establish not only the existence but also the expression level of hotspot mutations in the following oncogenes: PIK3CA, KRAS, NRAS, AKT1, BRAF, IDH1, CTNNB1, and IDH2. Of the 3,784 patients in this analysis, 720 (˜19%) were found to have mutations in the oncogenes listed above. Remarkably, only 38 (5.3%) of these patients had better than 90% expression (as compared to maximum expression) by RNA analysis, and 36 patients (5.0%) with identified hotspot mutations had no or low (<10% of maximum) expression. For example, mutations at position E545 in the PI3K protein encoded by the PIK3CA gene, which has been targeted by both pan-PI3K and mutant-targeted drugs in clinical trials, showed low or no expression in 12% (5/41) of breast cancer patients, and not a single patient showed relatively maximal (>90%) expression. Similarly of the 204 thyroid cancer patients with a BRAF V600 hotspot mutation, 7.5% (15/204) had low or no expression and none had relatively maximal expression. As is readily apparent, these findings illustrate that genetic mutations in gene panels (‘hotspots’) do not always result in protein expression. Given that many gene mutations were not expressed, it should be recognized that informed molecularly-driven clinical treatment decisions require insight into downstream protein expression and not just DNA alterations alone.

These findings and shortcomings prompted the inventors to integrate whole genome and RNA sequencing with quantitative (actually measured or inferred) proteomics to better inform clinical treatment selection. In a further computational omics study, over 50 unique tumors from primary and metastatic disease were selected for panomic tumor profiling. A cloud-based DNA and RNA supercomputing platform was developed to produce copy-number estimates, somatic variants, rearrangements, and RNA-abundance estimates from FFPE (formalin fixed paraffin embedded) biopsies. Pathway analysis was integrated with the findings of the whole genome DNA sequencing and RNA data to infer proteomics and predict drug targets. Quantitative and multiplexed proteomic analysis by mass-spectrometry from FFPE biopsies validated therapeutic targets at attomoles per micrograms of tissue.

Notably, the inventors discovered in this study that approximately 80% of all tumors had somatic events in heretofore known “actionable” genes, and multiple cases showed confirmation between predicted actionable genes and quantitatively increased protein expression. However, many mutations showed little or no expression at the transcriptomic level, which was confirmed by quantitative proteomic measurements in actual patient samples. Intriguingly, the inventors also observed genomic mutations and expression of proteins for which approved drugs are available, independent of anatomical tumor type. Thus, it should be appreciated that panomic analysis with pathway integration of a tumor in a first tissue may reveal druggable targets only approved and known for tumor treatment in a second, distinct tissue. Consequently, the inventors conclude that the molecular signature of a cancer patient is independent of the anatomical tumor type and, given that many gene mutations may not be expressed, informed clinical treatment decisions require insight into downstream protein expression in addition to DNA alterations.

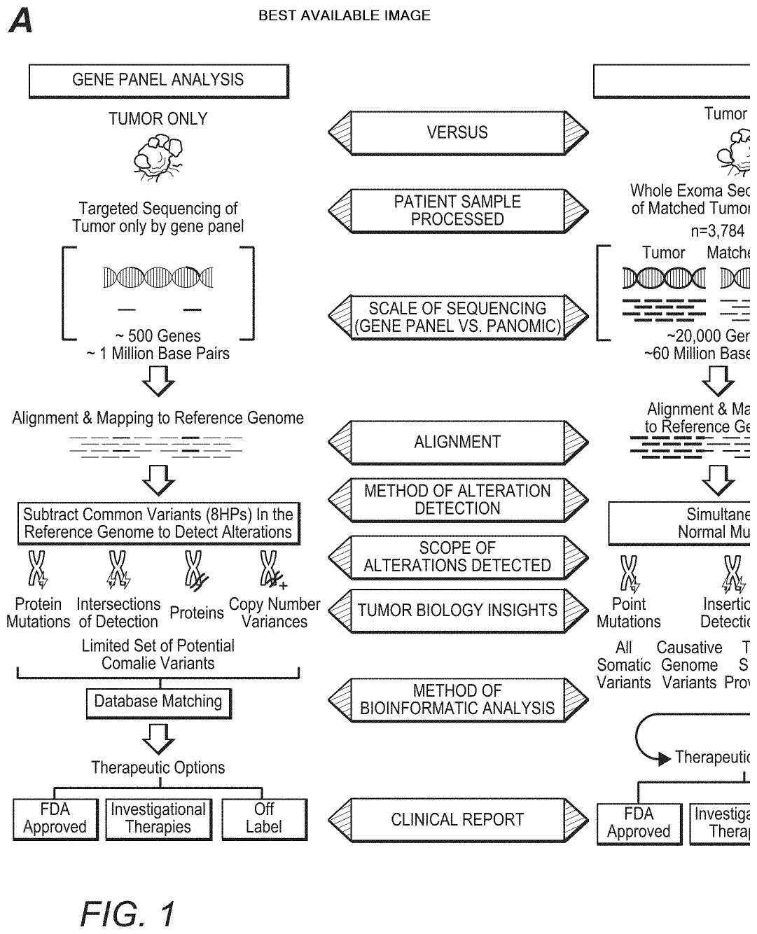

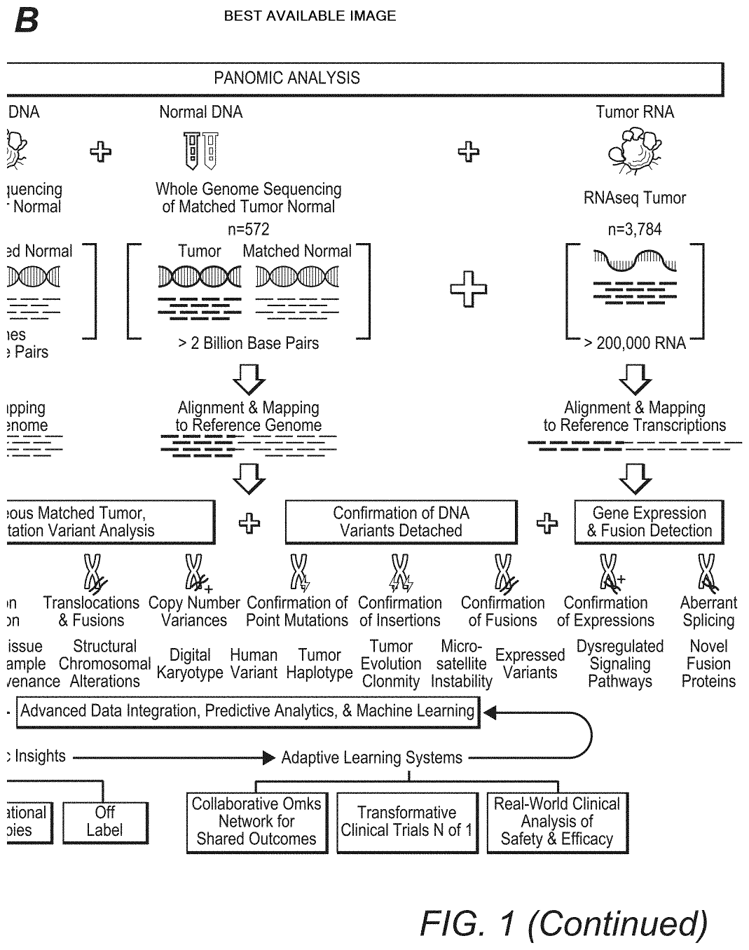

To address such shortcomings, the inventors now present integrated omics test systems and methods using matched tumor-normal samples, RNA sequencing and/or analysis of transcription level, combined with pathway analysis to so precisely define or rule out clinically actionable targets, potentially leading to better informed treatment selection. For example, in one aspect of the inventive subject matter, the inventors contemplate a method of calculating a treatment recommendation using omics information. Especially preferred methods include a step of obtaining, by an analysis engine, from an omics data base or sequencing facility (1) omic information of a patient, wherein the omic information is generated from genomic sequence information of a genomic sequence in a diseased and a matched normal sample, and (2) a transcription level for the genomic sequence in at least the diseased sample. In another step, the omic information and the transcription level for the genomic sequence in at least the diseased sample is used by the analysis engine in a pathway model to calculate a pathway activity of a pathway containing the genomic sequence, and in yet another step, the analysis engine identifies a druggable target based on the calculated pathway activity. The calculated pathway activity is then used to update or generate a patient record with a treatment recommendation. Alternatively, or additionally, the analysis engine classifies a tumor based on the calculated pathway activity, wherein the step of classifying is performed independent of tumor anatomy. The classification of the tumor is then used to update or generate a patient record with a treatment recommendation. An exemplary illustration comparing gene panel analysis against panomic analysis is provided in the work flow of FIG. 1. As can be readily seen from FIG. 1, numerous advantages can be realized in panomic analysis that are unavailable using conventional gene panel analysis.

To demonstrate the applicability and value of contemplated panomic test methods, the inventors analyzed DNA mutations (exome and whole genome), RNA mutations, transcription levels of RNA (and especially aberrant expression of RNA) of tumor and matched normal tissues, and performed a pathway analysis (using PARADIGM) to infer downstream protein alterations of patient matched tumor-normal samples from a large population of 3783 cancer patients across 18 tumor types. The precision, accuracy, and clinical value of the findings identified by the panomic test was then compared against the DNA mutation calls of a 328-gene panel of Table 1.

TABLE 1
328-Gene Panel

| | | | | | | | |
|---|---|---|---|---|---|---|---|
| ABL1 | CCND3 | ERG | GLI1 | LYN | NUP93 | SDHA | WISP3 |
| ABL2 | CCNE1 | ERRFI1 | GNA11 | LZTR1 | PAK3 | SDHB | WT1 |
| ACVR1B | CD274 | ESR1 | GNA13 | MAGI2 | PALB2 | SDHC | XPO1 |
| AKT1 | CD79A | ETV1 | GNAQ | MAP2K1 | PARK2 | SDHD | ZBTB2 |
| AKT2 | CD79B | ETV4 | GNAS | MAP2K2 | PAX5 | SETD2 | ZNF217 |
| AKT3 | CDC73 | ETV5 | GPR124 | MAP2K4 | PBRM1 | SF3B1 | ZNF703 |
| ALK | CDH1 | ETV6 | GRIN2A | MAP3K1 | PDCD1LG2 | SLIT2 | |
| AMER1 | CDK12 | EZH2 | GRM3 | MCL1 | PDGFRA | SMAD2 | |
| APC | CDK4 | FAM123B | GSK3B | MDM2 | PDGFRB | SMAD3 | |
| AR | CDK6 | FAM46C | H3F3A | MDM4 | PDK1 | SMAD4 | |
| ARAF | CDK8 | FANCA | HGF | MED12 | PIK3C2B | SMARCA4 | |
| ARFRP1 | CDKN1A | FANCC | HNF1A | MEF2B | PIK3CA | SMARCB1 | |
| ARID1A | CDKN1B | FANCD2 | HRAS | MEN1 | PIK3CB | SMO | |
| ARID1B | CDKN2A | FANCE | HSD3B1 | MET | PIK3CG | SNCAIP | |
| ARID2 | CDKN2B | FANCF | HSP90AA1 | MITF | PIK3R1 | SOCS1 | |
| ASXL1 | CDKN2C | FANCG | IDH1 | MLH1 | PIK3R2 | SOX10 | |
| ATM | CEBPA | FANCL | IDH2 | MLL | PLCG2 | SOX2 | |
| ATR | CHD2 | FAS | IGF1R | MLL2 | PMS2 | SOX9 | |
| ATRX | CHD4 | FAT1 | IGF2 | MLL3 | POLD1 | SPEN | |
| AURKA | CHEK1 | FBXW7 | IKBKE | MPL | POLE | SPOP | |
| AURKB | CHEK2 | FGF10 | IKZF1 | MRE11A | PPP2R1A | SPTA1 | |
| AXIN1 | CIC | FGF14 | IL7R | MSH2 | PRDM1 | SRC | |
| AXL | CREBBP | FGF19 | INHBA | MSH6 | PREX2 | STAG2 | |
| BAP1 | CRKL | FGF23 | INPP4B | MTOR | PRKAR1A | STAT3 | |
| BARD1 | CRLF2 | FGF3 | IRF2 | MUTYH | PRKCI | STAT4 | |
| BCL2 | CSF1R | FGF4 | IRF4 | MYB | PRKDC | STK11 | |
| BCL2L1 | CTCF | FGF6 | IRS2 | MYC | PRSS8 | SUFU | |
| BCL2L2 | CTNNA1 | FGFR1 | JAK1 | MYCL | PTCH1 | SYK | |
| BCOR | CTNNB1 | FGFR2 | JAK2 | MYCL1 | PTEN | TAF1 | |
| BCORL1 | CUL3 | FGFR3 | JAK3 | MYCN | PTPN11 | TBX3 | |
| BCR | CYLD | FGFR4 | JUN | MYD88 | QKI | TERC | |
| BLM | DAXX | FH | KAT6A | MYST3 | RAC1 | TERT | |
| BRAF | DDR2 | FLCN | KDM5A | NF1 | RAD50 | TET2 | |
| BRCA1 | DICER1 | FLT1 | KDM5C | NF2 | RAD51 | TGFBR2 | |
| BRCA2 | DNMT3A | FLT4 | KDM6A | NFE2L2 | RAF1 | TMPRSS2 | |
| BRD4 | DOT1L | FOXL2 | KDR | NFKBIA | RANBP2 | TNFAIP3 | |
| BRIP1 | EGFR | FOXP1 | KEAP1 | NKX2-1 | RARA | TNFRSF14 | |
| BTG1 | EMSY | FRS2 | KEL | NOTCH1 | RB1 | TOP1 | |
| BTK | EP300 | FUBP1 | KIT | NOTCH2 | RBM10 | TOP2A | |
| C11orf30 | EPHA3 | GABRA6 | KLHL6 | NOTCH3 | RET | TP53 | |
| C17orf39 | EPHA5 | GATA1 | KMT2A | NPM1 | RICTOR | TSC1 | |
| CARD11 | EPHA7 | GATA2 | KMT2C | NRAS | RNF43 | TSC2 | |
| CBFB | EPHB1 | GATA3 | KMT2D | NSD1 | ROS1 | TSHR | |
| CBL | ERBB2 | GATA4 | KRAS | NTRK1 | RPTOR | U2AF1 | |
| CCND1 | ERBB3 | GATA6 | LMO1 | NTRK2 | RUNX1 | VEGFA | |
| CCND2 | ERBB4 | GID4 | LRP1B | NTRK3 | RUNX1T1 | VHL | |

Utilizing a massively parallel computing platform, a large-scale patient sequencing data set of patient matched tumor-normal samples was analyzed from The Cancer Genome Atlas (TCGA) CG-Hub database (see URL cghub.ucsc.edu). Whole exome sequencing (WES) data RNA sequencing data (sequence and transcription level) and whole genome sequencing (WGS) data were available for 3783 and 572 patients, respectively. DNA sequencing data were processed using ‘Contraster’, following methods as described in US2012/0059670A1 and US20120066001A1, and gene panel analysis was limited to the 328 genes in Table 1. Panomic analysis and gene-panel analysis were compared to determine high expression of called mutations in the whole exome versus gene panel, called mutations with low/no expression in the gene panel, and high expression of actionable non-mutated (noncalled) genes in the gene panel.

RNA data were used to confirm the presence of alterations in transcripts and classified variants into high-expressed (>0.9 allele fraction) and low-expressed (<0.1 allele fraction) variants. RNA expression values were obtained from level 3 data transcript per million (TPM) values. High expression of RNA sequences was defined as TPMs with a Z-score≥3, with the normal distribution defined per gene as the set of all adjacent normal samples across tissues. PARADIGM was used to reveal shared pathways among patients who had genomic and transcriptomic data. Copy-number alterations were derived from segmented data produced by ‘Contraster’. Transcriptomic data were normalized to a pool of normal samples across all tissues, with transcript abundance identified as up- (or down-) regulated when falling above the upper (or below the lower) 5th percentile for each gene. Clustering of PARADIGM activity values was performed using a custom version of Cluster 3.0. Breakpoint analysis was restricted to 572 patients with WGS data, and microsatellite instability (MSI) was determined for 3783 patients with WES data. The percentage of matching or compatible genotypes were used as a similarity metric.

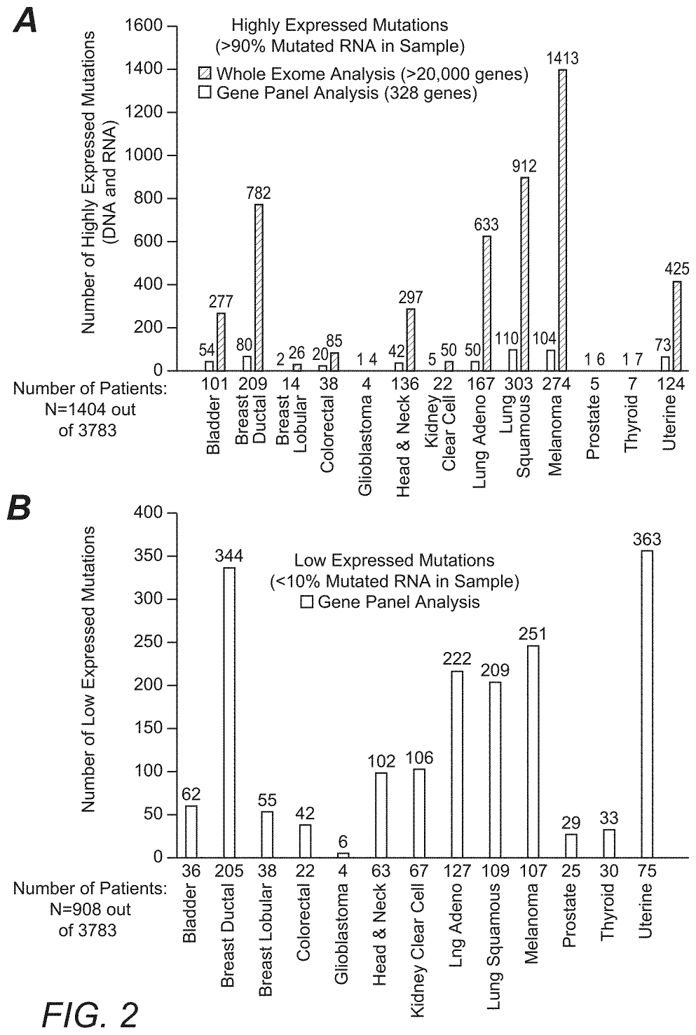

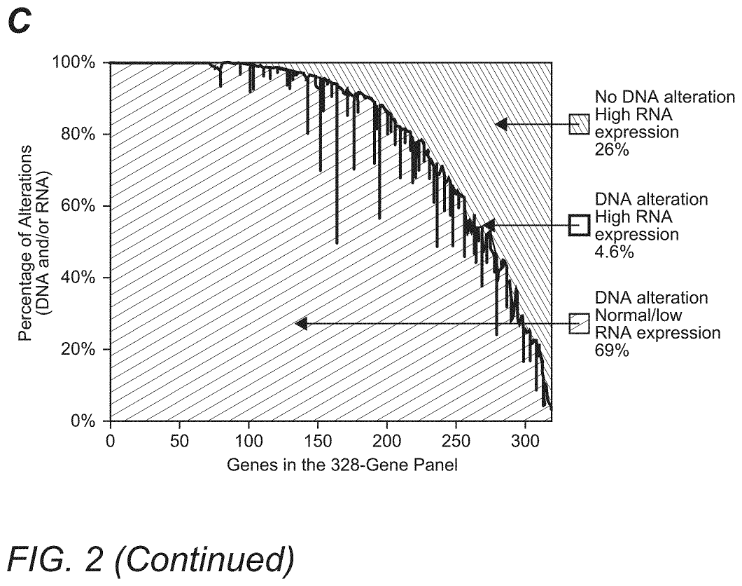

Using the above methods, the inventors noted that of 3783 patients, 1404 (37%) had mutations that were highly expressed; patients with melanoma, lung, breast, and uterine cancers had the most highly expressed mutations as can be taken from FIG. 2, Panel A. A substantial difference in the number of detected highly expressed mutations was observed by panomic (whole exome) analysis versus gene-panel analysis. Here, only 543 (11%) of highly expressed mutations were identified by gene-panel analysis versus 4917 identified by panomic analysis, an 89% missed rate of detection when limiting the test to a gene panel as can be seen in FIG. 2, Panel A. Among 274 patients with melanoma, more than a thousand highly expressed mutations were identified by panomic versus gene-panel analysis (1404 versus 104), representing a 13.5-fold improvement in detection. While quantitatively significant, the additionally detected mutations are even more important when taken into account in a subsequent pathway model.

Regarding the transcriptomic analysis to exclude false-positive mutations (i.e., mutations present in the genome, but not transcribed to RNA), the inventors used a filter for the 328 genes included in the gene panel to determine the number of patients who had tumor mutations, but low RNA expression. As can be seen from FIG. 2, Panel B, of 3783 patients, 908 (24%) had panel mutations expressed in <10% RNA transcripts and 181 (4.8%) had mutations expressed in <1% RNA transcripts. Consequently, identification of mutations in the gene panel alone without transcriptomic information will lead to false-positive calls and so expose patients to treatment that is not likely to be effective. For example, among patients with melanoma, 107 patients had mutations expressed in <10% RNA transcripts and 19 patients had mutations expressed in <1% RNA transcripts. Among patients with ductal or lobular breast cancer, 243 patients had mutations expressed in <10% RNA transcripts and 62 patients had mutations expressed in <1% RNA transcripts. Focusing on individual hotspot mutations, 328 patients had mutations in BRAF V600E, of whom 15 (4.6%) had <10% mutated RNA. Additionally, of the 81 patients who had PIK3CA E545 mutations, 11 (13.6%) had <10% mutated RNA (data not shown). Clearly, these results emphasize the high likelihood of ineffective targeted treatment where the target is not being expressed when only DNA sequence information is considered.

Likewise, with respect to the detection of highly expressed DNA alterations in the gene panel, the inventors examined associations between RNA expression and DNA alterations for each gene in the panel, and results are illustrated in FIG. 2, Panel C. Only 4.6% of genes with positively called DNA mutations/amplifications were associated with high RNA expression, while 69% of genes with DNA mutations/amplifications had low/normal RNA expression. These data demonstrate that a correct call (DNA mutation with high RNA expression) is made for <5% of genes when limiting the analysis to tumor DNA in the absence of RNA sequence data. Still further, the inventors also found that 26% of genes in the gene panel had no evidence of DNA mutations/amplifications but had high RNA expression, a 26% false-negative rate, which can only be identified by a comprehensive panomic test.

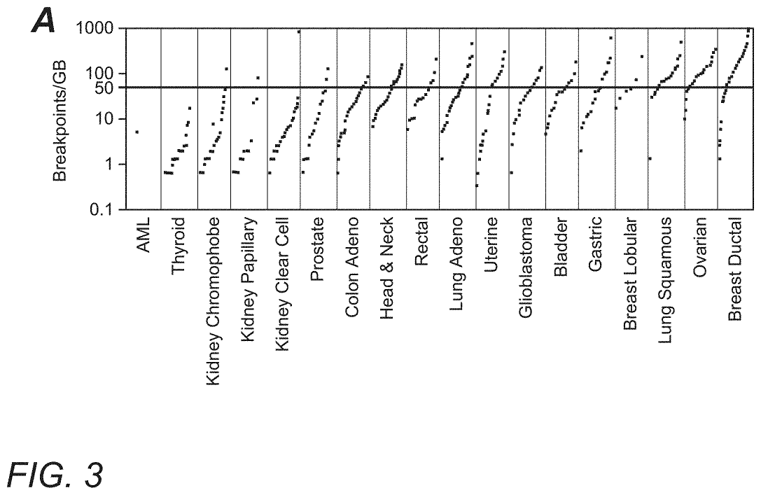

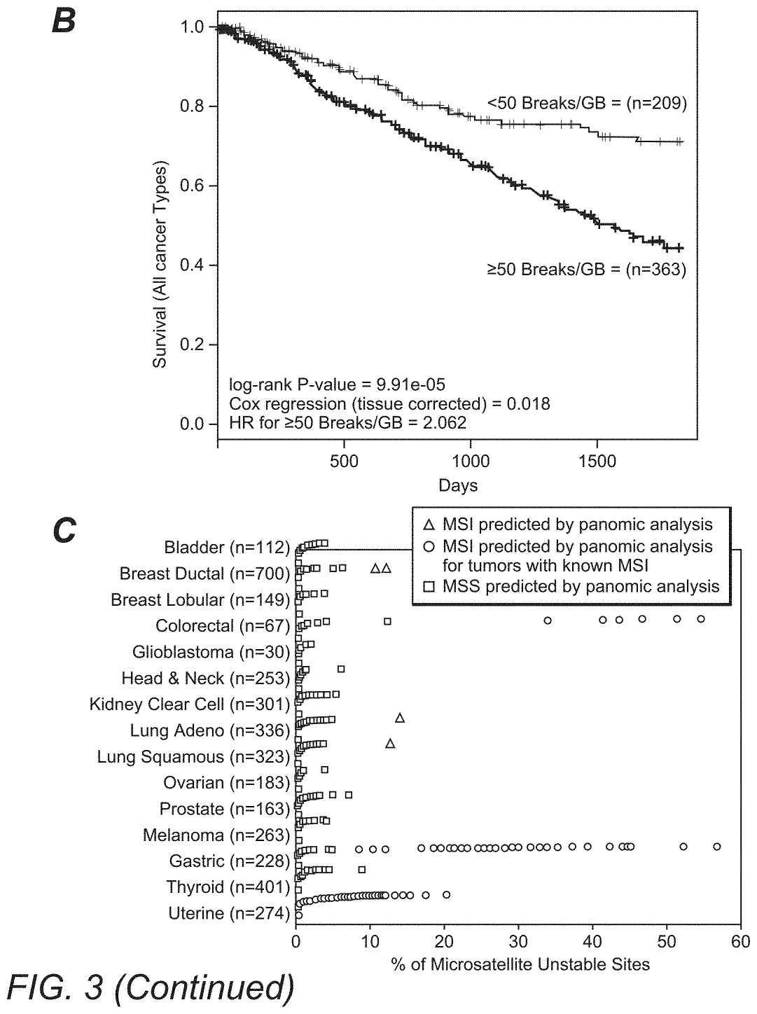

As a further unexpected result from using analysis of tumor tissue with matched normal control tissue, the inventors discovered that frequency of breakpoints closely correlated to MSI (Microsatellite Instability). Here, genomic re-arrangement profiles of patients for whom there were WGS data revealed wide patterns of genomic rearrangements as can be seen from FIG. 3, Panel A. Notably, a subset of tumors contained a large number of breakpoints (>50 breakpoints per gigabase), with the most being observed in ductal breast cancer and uterine cancer. Of the 572 total patients with breakpoint data, those with >50 breakpoints/gigabase (n=363) versus <50 breakpoints/gigabase (n=209) had significantly worse overall survival (P<0.001); overall survival remained worse when controlled for tumor type (P=0.018) as is shown in FIG. 3, Panel B. It should be appreciated that such classification is not only of statistical value but also allows to inform the clinician of potential treatment options: MSI status is routinely used to diagnose Lynch Syndrome among gastric and colorectal cancer patients, and often to predict responsiveness to 5-fluorouracil. A tumor versus matched normal analysis was used to predict MSI percentages for 3783 patients. As can be taken from the prediction results in FIG. 3, Panel C, MSI-High status was perfectly discriminated in colorectal and gastric cancers (1.0 area under the curve [AUC]), and was nearly perfect in uterine cancer (0.98 AUC). MSI was confirmed by the clinical annotations obtained with the samples. Using the above approach, the inventors identified a high percentage of MSI in colorectal, gastric, and uterine cancers as well as in lung cancer and ductal breast cancer, which has not been previously reported. Moreover, MSI can in all or almost all cases not be detected by targeted sequencing, and current tests for MSI use PCR-based methodologies, which may yield false-positive results in 10 to 15% of patients. Unlike PCR tests, contemplated systems and methods perfectly discriminated MSI in colon cancer and gastric cancer.

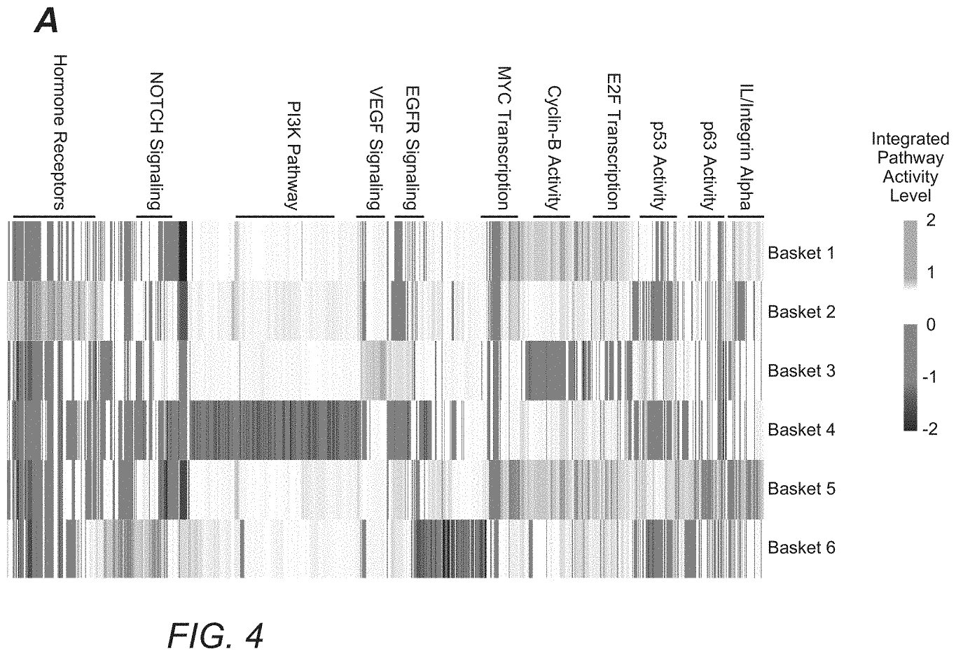

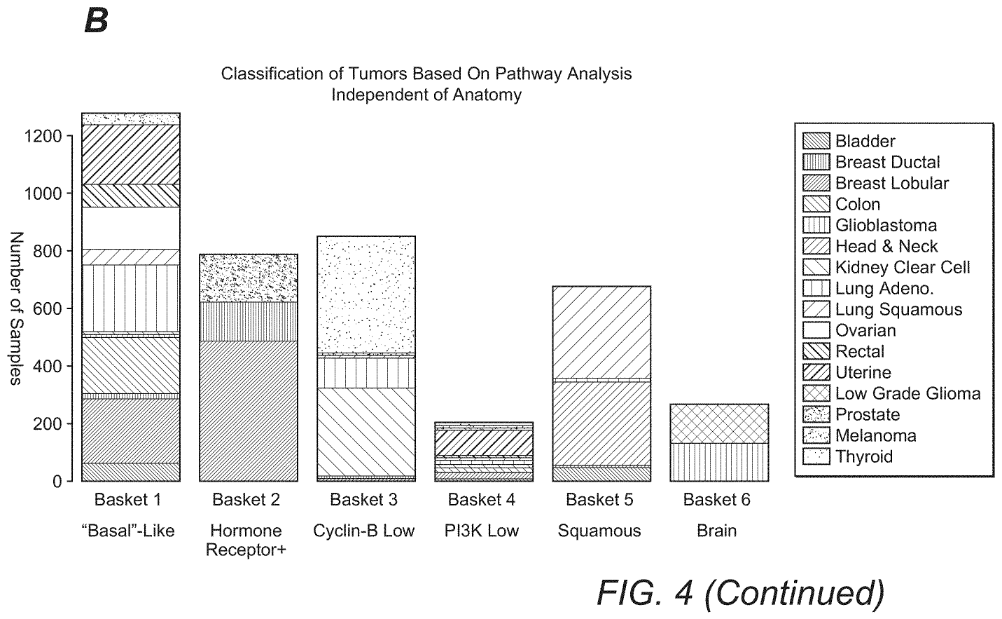

The inventors further discovered that classification of tumors could also be performed based on pathway analysis in a manner that was entirely independent of the anatomy of the tumor (e.g., agnostic to the origin/type of cancer). More particularly, when the genomic and transcriptomic information was integrated into a pathway model (here: PARADIGM: pathway recognition algorithm using data integration on genomic models as, for example, described in WO2011/139345A2 and WO2013/062505A1), all patients could be grouped into six major ‘baskets’ as is exemplarily shown in FIG. 4, Panel A. Here, the basket of “basal”-like tumors (basket 1) share high levels of MYC transcription, E2F transcription, and cyclin-B activity. One basket of up-regulated hormone receptor tumors included primarily patients with breast or prostate cancer (basket 2). Lung adenocarcinoma, kidney clear cell cancer, and thyroid cancer all had low cyclin-b activity but increased VEGF activity compared to the other tumors (basket 3). Another heterogeneous set of tumors contained a signature of decreased PI3K activity and increased p53 pathway activity (basket 4). A clear squamous subtype had high MYC, cyclin-B, and E2F activity, similar to the “basal”-like basket; however, it had increased activity in the p53 and interleukin pathways (basket 5). The tissue-specific “brain” basket (basket 6) had some of the lowest activities overall, but it showed marked increase in NOTCH signaling pathways.

As can be seen from the above, the integration of WGS, WES, from matched tumor-normal patient samples with RNA data and pathway analysis is important to more accurately and comprehensively capture actionable information and precisely inform treatment selection for patients with cancer. Indeed, patients with tumors belonging to one ‘basket’ are suitable for and likely responsive to treatment with drugs targeting the signaling pathways common to those baskets, irrespective of the tumor type or origin. An exemplary grouping of tumors to ‘baskets’ is shown in FIG. 4, Panel B.

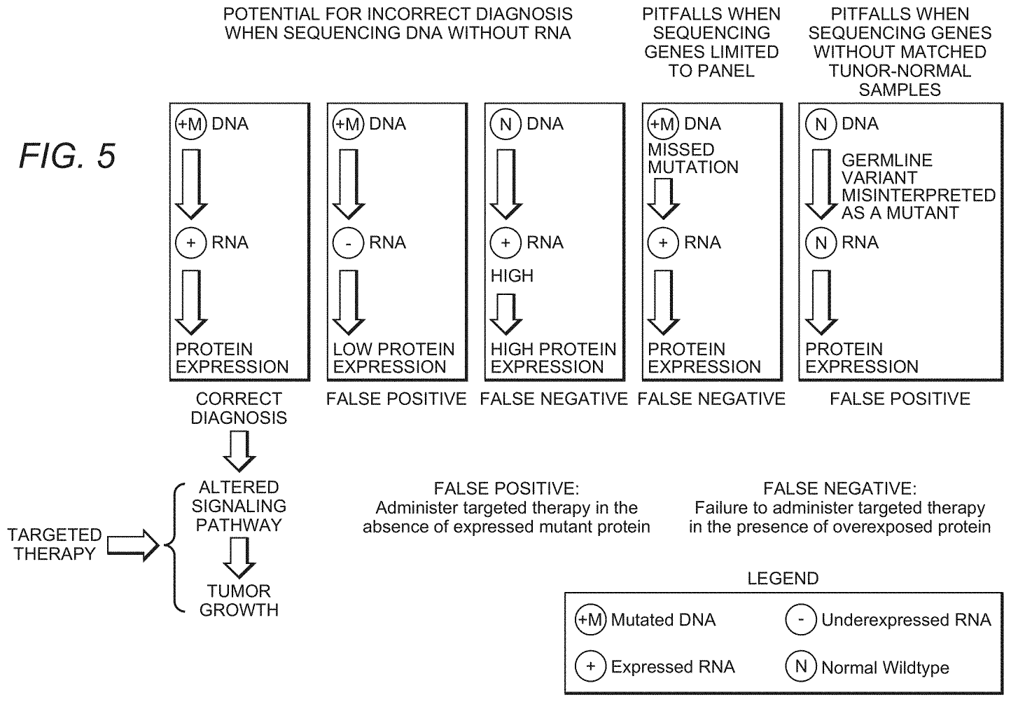

The use of gene panels, which analyze only DNA, assumes that gene mutations result in changes in protein expression and dysregulated cell signaling, and that treatment decisions can be based solely on the presence of specific gene alterations. The inventors' analysis demonstrates that this assumption is not necessarily correct. Indeed, DNA alterations associated with high RNA expression, DNA alterations associated with low RNA expression, high RNA expression not associated with DNA alterations, and mutations that may be missed by limiting the number of analyzed genes all materially and significantly factor in whether or not a treatment with a particular drug may be suitable. Therefore, the use of panels limited to a few hundred genes, sequencing DNA without RNA, and sequencing tumor DNA without matched tumor-normal samples may, therefore, yield false-negative or false-positive results potentially leading to inappropriate treatment decisions for patients. The risk of incorrect diagnosis or treatment action is schematically illustrated in FIG. 5. As an example, RET was mutated/amplified but not highly expressed in 98 patients, mutated/amplified and highly expressed in 7 patients, and highly expressed without mutation/amplification in 333 patients.

Regarding highly expressed and actionable somatic mutations that may be missed using a gene panel alone, the inventors identified 4 patients with melanoma and 1 patient with breast cancer who had mutations in the S6 kinase gene and expressed high levels of S6 kinase RNA. However, S6 kinase is not included in any currently known gene panel. S6 kinase is part of the mTOR signaling pathway and is a potential predictive biomarker for response to everolimus, an inhibitor of mTOR. While genes are added to panels as discoveries are made, patients tested using panels will not benefit from future scientific advances without undergoing retesting. Conversely, the inventors also discovered that an integrated panomic approach can yield false-positive results. Among 328 patients with BRAF mutations and 81 patients with the PI3-kinase (PI3K) E545 mutation, 4.6% and 13.6%, respectively, had <10% mutated RNA. Therefore, patients with low BRAF expression may not benefit from drugs targeting BRAF, and enrollment of patients with low PI3K expression into clinical trials of anti-PI3K agents may not be appropriate. In the phase 2 FERGI trial, the PI3K inhibitor pictilisib failed to improve outcomes in patients with ER+ metastatic breast cancer with or without PIK3CA-mutant tumors. In addition, patients with HER2+ breast cancer tumors harboring PIK3CA mutations responded poorly to trastuzumab. For both studies, it is possible that poor responders had little or no expression of the mutated gene. As such, the inventors also contemplate use of the systems and methods presented herein for selection of candidates for clinical trials of drugs that target a specific drug or signaling pathway.

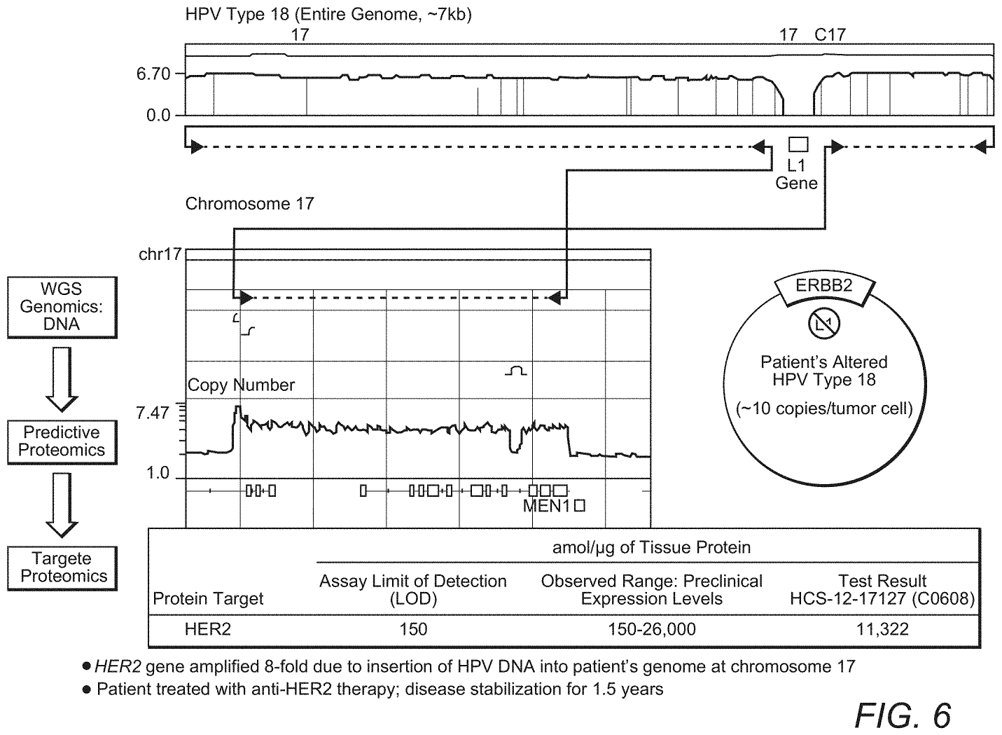

In addition, panomic analysis according to the inventive subject matter presented herein is also beneficial where structural and/or functional rearrangement of portions of the genome occur. For example, as is shown in FIG. 6, a rearrangement between the patient's genome and the genome of HPV18 occurred such that the patient's ERBB2 gene translocated to the viral genome, which was functionally amplified to about 10 copies per tumor cell after integration of the virus genome into the patient's genome. In this example, it should be appreciated that only a tumor-matched normal sample approach could identify such translocation and that only genomic and transcriptomic analysis revealed a possible treatment option using anti-HER2 therapy that provided disease stabilization for 18 months in a terminally ill patient.

Most typically, the genomic sequence information comprises DNA sequence information that is obtained from the patient, most preferably from the diseased tissue and matched healthy tissue; from blood for example. For example, suitable genomic information may include exome sequence information, whole genome sequence information, or portions thereof where only specific portions need to be identified. There are numerous methods of partial and complete genome sequencing known in the art, and all of them are deemed suitable for use herein. However, especially preferred methods include next generation sequencing and high-throughput sequencing methods (e.g., ABI/Life Technologies SOLiD; Helicos HeliScope; Illumina Genome Analyzer; Roche/454 GS FLX Titanium; Sanger capillary sequencing). Consequently, numerous data formats will also be deemed appropriate, however, particularly preferred data formats include raw various formats, FASTA, SAM, and BAM formats. Where diseased and healthy tissues are analyzed from a single patient, BAM format is particularly preferred.

With respect to the analysis of tumor and matched normal tissue of a patient, numerous manners are deemed suitable for use herein so long as such methods will be able to generate a differential sequence object or other identification of location-specific difference between tumor and matched normal sequences. However, it is especially preferred that the differential sequence object is generated by incremental synchronous alignment of BAM files representing genomic sequence information of the diseased and the matched normal sample. For example, particularly preferred methods include BAMBAM-based methods as described in US2012/0059670A1 and US20120066001A1.

Likewise, with respect to RNA sequence information, it is contemplated that all manners of RNA sequencing are deemed suitable for use herein. However, especially preferred methods include those that are based on isolation and/or reverse transcription of polyadenylated RNA. Moreover, suitable data formats for RNA will include various raw formats, FASTA, SAM, and BAM formats. Moreover, it should also be noted that where the RNA sequence information is in BAM format, omic analysis may be performed using a BAMBAM in which germline DNA, somatic DNA, and RNA can be concurrently processed. In addition, it should also be appreciated that panomic analysis as presented herein may also include protein quantification and activity determination of selected proteins. Such proteomic analysis can be performed from freshly resected tissue, from frozen or otherwise preserved tissue, and even from FFPE tissue samples. Most preferably, proteomics analysis is quantitative (i.e., provides quantitative information of the expressed polypeptide) and qualitative (i.e., provides numeric or qualitative specified activity of the polypeptide). Example suitable techniques for conducting such quantitative proteomic analysis on tissue samples are describe in U.S. Pat. Nos. 7,473,532; 8,455,215; and 9,163,275, and are available via OncoPlex Diagnostics (see URL www.oncoplexdx.com).

Therefore, in one aspect of the inventive subject matter, the inventors also contemplate a method that identifies situations in which omics information would give rise to false positive or false negative results. More specifically, where a differential sequence object is generated from genomic sequence information of a diseased and a matched normal sample, and where the RNA transcription level for the corresponding genomic sequence is available, an analysis engine may apply a threshold level to the transcription information. The differential sequence object may then be annotated as (a) false positive upon determination that the transcription level is below the threshold level or (b) true positive upon determination that the transcription level is above the threshold level. Where tumor and matched normal have no difference for a specific sequence, and where the level is above the threshold level, the specific sequence may be annotated as false negative. Thus, it should be appreciated that omics analysis as presented herein will not only provide sequence comparisons for a specific sequence, but will also provide an indication of whether or not that specific sequence is likely to be a determinant for identification of a target suitable for pharmaceutical intervention.

With respect to the pathway analysis, it is generally preferred that the analysis uses an a priori established model that, based on input data, will yield calculated output data with respect to pathway elements and/or pathway activity (e.g., pathway utilization relative to normal). Suh models may be entirely empirical, or based on machine learning, or based on actual and inferred data. Moreover, it should also be recognized that pathway analysis may utilize ensembles of models from which one or more are selected to calculate the output data. In one preferred aspect of the inventive subject matter, patient and tumor specific pathway analysis is performed using PARADIGM as disclosed in WO2011139345A2 and WO2013062505A1.

As can be readily seen from the above, contemplated systems and methods will require substantial demands on a computing infrastructure where treatment recommendations are to be provided for more than one patient at a time. Therefore, the inventive subject matter provides apparatus, systems and methods in which a high throughput analysis system is able to process many patient tissue samples in parallel in order to generate hundreds or even thousands of clinical reports. Contemplated systems include at least one multi-lane sequencing device able to process tissue samples from multiple patients at the same time. The multi-lane sequencing device can process both a normal tissue sample and a disease tissue (e.g., tumor tissue, etc.) in a common run, possibly where the tissue samples are placed in a single flow cell lane. The sequencing devices are further configured to generate sequence data (e.g., whole genome sequences, etc.), exome sequences (e.g., exons from the genome sequence, etc.), and RNA sequences for both the normal tissue and the disease tissue. Further, the sequencing devices are also configured to enrich the exome sequence and RNA sequences relative to the genomic sequences by at least a factor of five.

Contemplated high-throughput analysis systems further include a modeling computer system able convert the sequence data from the sequencing devices into actionable clinical data. The modeling computer system comprises a processor (e.g., Intel® i7®, AMD® Opteron®, GPU, CPU, etc.), a memory (e.g., flash, RAM, HDD, SSD, SAN, RAID, etc.), and modeling and report engine. The modeling and reporting engine is executable on the processor according software instructions stored in the memory where the engine is able to store the sequence data in the memory, perhaps in a SAM, BAM, VCF, BAMBAM, or other format. The engine further stores one or more sequence-based treatment models in the memory. Example treatment models could include drug response models, treatment outcome models, pathway expression models, or other types of clinically relevant models. Each of the models is configured to generate patient-specific clinical report data. Thus, the engine is able to execute the sequence-based treatment models on one or more of the genome sequence data, the exome sequence data, and the RNA sequence data of the normal and disease tissues in order to generate the patient-specific clinical data. For example, the patient-specific clinical report data could include a predicted drug response of the tumor tissue relative to the normal tissue as determined by multiple genome-based drug response models a priori generated from cohort data. The engine compiles the patient specific clinical data into a clinical report, which can then be presented on an output device (e.g., secured “omics” browser device, printer, computer, display, etc.).

It should be noted that any language directed to a computer should be read to include any suitable combination of computing devices, including servers, interfaces, systems, databases, agents, peers, engines, controllers, modules, or other types of computing devices operating individually or collectively. One should appreciate the computing devices comprise a processor configured to execute software instructions stored on a tangible, non-transitory computer readable storage medium (e.g., hard drive, FPGA, PLA, solid state drive, RAM, flash, ROM, etc.). The software instructions configure or program the computing device to provide the roles, responsibilities, or other functionality as discussed below with respect to the disclosed apparatus. Further, the disclosed technologies can be embodied as a computer program product that includes a non-transitory computer readable medium storing the software instructions that causes a processor to execute the disclosed steps associated with implementations of computer-based algorithms, processes, methods, or other instructions. In some embodiments, the various servers, systems, databases, or interfaces exchange data using standardized protocols or algorithms, possibly based on HTTP, HTTPS, AES, public-private key exchanges, web service APIs, known financial transaction protocols, or other electronic information exchanging methods. Data exchanges among devices can be conducted over a packet-switched network, the Internet, LAN, WAN, VPN, or other type of packet switched network; a circuit switched network; cell switched network; or other type of network.

As used in the description herein and throughout the claims that follow, when a system, engine, server, device, module, or other computing element is described as configured to perform or execute functions on data in a memory, the meaning of “configured to” or “programmed to” is defined as one or more processors or cores of the computing element being programmed by a set of software instructions stored in the memory of the computing element to execute the set of functions on target data or data objects stored in the memory.

It is noted that the inventive subject matter enables construction or configuration of a computing system to operate on vast quantities of digital data, beyond the capabilities of a human. Although the digital data represents one or more aspects of genomic sequences, it should be appreciated that the digital data is a representation of one or more digital models of a genome, not the genome itself. By instantiation of such digital genomic models as well as treatment models in the memory of the computing devices, the computing devices are able to manage the digital data or models in a manner that provides utility to a user of the computing device that the user would lack without such a tool.

Typically, whole genome sequencing is performed with relatively high depth, 50× reads for example. An increase in reads further increases the amount of time required to complete processing. The inventors have appreciated that, even if the whole genome sequence (WGS) is available, the WGS does not necessarily provide complete clinically relevant information. The inventors have further appreciated that the expression details of the WGS have a greater impact on patient health than just the WGS itself. Still further, the inventors have conceived of a system that is able to process vast numbers of patient tissue samples and generate clinically relevant reports based on the expression details thereby placing significant personalized information in the hands of a treatment decision maker in a timely fashion; days instead of weeks.

Rather than focusing on just a WGS sequence itself, the inventors contemplate in one approach to emphasize exome sequences and RNA sequences relative to the WGS. This approach, especially when coupled with treatment outcome information, provides greater and more accurate insight into how a patient is likely to respond to treatment because it directly couples to the expression of genes rather the merely indicating presence of or problems with genes. The disclosed approach emphasizes exome sequences and RNA sequences relative to the WGS by enriching their depth relative to the WGS reads. For example, WGS might comprise 10× to 15× reads while exome sequence or RNA sequences might include 400× reads.

The disclosed system preferably comprises one or more multi-lane sequencing devices and a modeling computer system. It should be appreciated that the disclosed system is able to create patient tissue analysis pipeline that results in clinical reports where each stage of the pipeline can be individually managed to ensure a high duty cycle of processing.

In some embodiments, the multi-lane sequencing device is able to process eight, 16, or more samples via multiple flow cell lanes. An example multi-lane sequencing device supporting eight lanes that could be configured for use with the inventive subject matter includes the Illumina® HiSeq 4000. More than one multi-lane sequencing device can operate in parallel so that samples from 100s, 1000s, or more of patient tissues samples can be run at the same time. For example, the disclosed infrastructure could include 10 or more than 100 multi-lane sequencing devices. Assuming that each sequencing device could support eight to 16 flow cell lanes, such an infrastructure could process 80 to 1600 lanes in parallel and then generate 1000s of reports on a daily basis.

The multi-lane sequencing devices are configured to sequence one or more patient's normal and disease tissues during a common run. For example, the patient's normal and disease tissue samples can be placed in a single common flow cell lane. This ensures timely results for each type of tissue and ensures timely comparison between the two tissues.

The multi-lane sequencing device or devices is further configured and/or operated to generate a genome sequence, exome sequence, and RNA sequence of both the patient's normal tissue and disease tissue (e.g., tumor tissue, etc.). Further, the devices are configured and/or operated to enrich the exome and RNA sequences relative to the genome sequence by at least a factor of five so that the expression related information is more emphasized. In at least some embodiments, the genome (WGS or WES) sequence data would have less than 20× reads, with possibly less than 10× reads. Further, in other embodiments, there can be a difference in sensitivity between the normal tissue genome sequence and the disease tissue genome sequence. For example, the disease tissue genome sequence might comprise 15× reads while the normal tissue might comprise 10× reads. The genome sequence can include a whole genome if desired.

It should be appreciated the exome sequence is considered to include selected portions of the whole genome sequences that reflect exons, mRNA, and alternate splice variants of mRNA rather than the genome sequence itself. Thus, the exome sequence, although represented by portions of DNA, is considered to be distinct from the genome sequence in general. The multi-lane sequencing device is configured/operated to further enrich the exome sequence by ensuring that it comprises at least 150× reads, 200× reads, 300× reads, or even 400× reads or more. In a similar vein, the sequencing device ensures that the RNA sequences comprise at least 150× reads, 200× reads, 300× reads, or even 400× reads or more. Therefore, the exome and/or RNA sequences are considered to be enriched by at least a factor of five, 10, 15, 30, or 40 or more relative to the genome sequence. By focusing on the expression information rather than WGS, the disclosed system is able to generate more accurate and actionable clinical reports.

The output genome sequence, exome sequence, and RNA sequence data can be further processed as part of the analysis/clinical reporting pipeline. For example, the sequence data or portions thereof can be annotated with additional metadata to facilitate down-stream processing. The annotations can include patient information, indications of where higher depths exist, prioritized areas to process, quality scores (e.g., estimated probability of a base being wrong), types of models or analysis to be conducted on the sequence data (e.g., apply trained classifiers to the sequence data, back propagating of the sequence data into machine learning system, etc.), or other information. Especially suitable methods of annotating and transmitting the sequence data can be found in US 2015/0134662.

The infrastructure contemplated herein also includes one or more modeling engines that include at least one memory, at least one processor, and a modeling and reporting engine configured to execute on the processor according to software instructions stored in the memory. The modeling and reporting engine can store the genome sequence, exome sequence, and RNA sequence in the memory according to one or more data formats. Example formats include SAM, BAM, BAMBAM, VCF, GAR, or other formats to facilitate processing or transport. BAMBAM format is considered advantageous because it captures differences between normal and disease tissues rather than requiring a complete genome sequence; thereby reducing memory foot print and increasing processing speed. Additional details regarding use of BAMBAM are described in US2012/0059670 and US2012/0066001.

The modeling and reporting (analysis) engine further stores one or more sequence-based treatment models in the memory. The treatment models can take on a broad spectrum of model types (e.g., clustering models, regression models, classifiers, etc.) that convert sequence data (e.g., genome sequence, exome sequence, RNA sequence, etc.) to clinically relevant data. For example, one type of treatment model could include a genome-based drug response model where the model has been a priori trained on cohort genome and treatment outcome data. Thus, when the patient's current sequences (i.e., normal and/or diseased tissue sequences) are provided to the drug response model, the drug response model would generate a predicated response outcome for the corresponding drug. Other types of models include a fully trained treatment outcome model, a pathway expression model, a PARADIGM model, or other types of models. Creation and use of outcome models are described more fully in international patent application publication WO 2014/193982. Creation and use of PARADIGM and pathway models are described in U.S. patent application publication US2012/0041683 and US2012/0158391. It should be appreciated that the modeling and reporting engine could leverage 100s, 1000s, 10000s, or even 100000s or more models to generate clinical report data.

The modeling and reporting engine leverages the sequence-based treatment models to generate patient-specific clinical report data. The type of report data depends on the nature of the corresponding model as alluded to above. Examples of report data that can be generated include predicted treatment responses, survivability, treatment protocols, potential drugs to be used in treatment, or other types of data. Further, the report data could comprise high level data generated from the ensemble of models leveraged in the analysis; accuracy of the models in aggregate for example. Such information is useful in ranking likely relevance of the report data.

The modeling and reporting engine compiles the patient-specific report data in order to generate a clinical report that can be the delivered to a designated stakeholder or a decision maker. For example, the report data could include metadata added during an annotation and transport step and that indicates the patient's name as well as their oncologist name. When the report is generated, it can be sent or routed to the oncologist for consumption by an output device (e.g., cell phone, tablet, desktop computer, printer, etc.). In some embodiments, the clinical report is packaged for display on a secured “omics’ browser devices, possibly based on the devices as discussed in US 2015/0310228.

The disclosed infrastructure is scalable to handle hundreds of thousands to millions of patients via a processing and analysis pipeline. The present infrastructure as disclosed allows for analysis of a single patient's normal and disease tissue and generation of a clinical report in about four days; three days for sequencing and one day for analysis via the modeling computer system. Still, when many patients are processed in parallel across multiple flow cell lanes, multiple sequencing devices, and across multiple facilities; the desired large scale processing of massive numbers of patients can be achieved.

In order to ensure a high duty cycle across portions of the architecture, the patient data can be staggered across the sequencing devices. For example, to start the overall system, one third of the sequencing devices can start processing on the first day. Another third of the sequencing devices starts sequencing on the second day. A final third of the sequencing devices start on the third day just as the initial results from the first third are generated. The results are then sent to the modeling and reporting engine for analysis. This approach ensures that the modeling engine is kept at full capacity, running efficiently, and has minimal down time.

Another interesting aspect of the disclosed approach is that the patient's tissue sequence data and clinical outcomes can be directed or routed to a side channel for model training and/or creation. The patient's data can be archived and used to create new, patient-specific models that can be individually stored and annotated with metadata describing the nature of the patient's health or state (e.g., type of disease, demographics, treatment given, etc.). The approach provides for one or more stakeholders to create a cohort of patient data where members in the cohort have common attributes. For example, the members of the cohort might have the same gender, demographic profile, geo-location, treatment protocols, prescribed drugs, treatment outcomes, genomic profiles, or other factors. The genomic data for the cohorts along with known treatment outcomes can be used to create new prediction models as discussed in WO 2014/193982.

Yet another interesting aspect of the inventive subject matter includes empirically measuring the relative merits of each type of sequencing data to determine the most efficient processing for a type of clinical report. Thus, the relative enrichment factors could be adjusted to decrease sequencing type while also increasing the accuracy or relevancy of the clinical report data.

Thus, it should be appreciated that mutations in genes targeted by drugs approved based on anatomy are prevalent in other cancers independent of tissue type, and identification of such mutations and associated pathways may open treatment options heretofore not realized in anatomy-based treatments. Thus, it should be recognized that not only mutational information, but also knowledge of expression levels for mutated and non-mutated genes matter, and can even be used as predictive tool (e.g., quantitative measurement of HER2>2200 amol/μg is predictive of longer survival). In addition, and where available, quantitative protein expression analysis will further improve treatment prediction. For example, a panomic platform integrating genomic sequencing with quantitative protein expression analysis informed effective treatment for a patient with end-stage cervical cancer with a drug that was not approved in that tissue type. Consequently, it should be recognized that precision cancer medicine will require reclassification of cancers based on their molecular profile and not on tissue type.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise. Where the specification claims refers to at least one of something selected from the group consisting of A, B, C . . . and N, the text should be interpreted as requiring only one element from the group, not A plus N, or B plus N, etc.

1-14. (canceled)

15. A high throughput computer-based genomic analysis system for reducing false positives and negatives, comprising:
at least one multi-lane sequencing device configured to sequence at least one patient's normal tissue and diseased tissue in a common run;
wherein the at least one sequencing device is further configured to generate a genome sequence, an exome sequence and an RNA sequence of the normal and disease tissues by sequencing a genome, an exome, and RNA of the tissues; and
wherein the exome sequence and RNA sequence are enriched relative to the genome sequence by at least a factor of five; and
a modeling computer system comprising:
at least one processor;
at least one memory; and
a modeling and reporting engine executable on the at least one processor:
according to software instructions store in the at least one memory and configured to:
store the genome sequence, the exome sequence, and the RNA sequence of the normal tissue and the diseased tissue of at least one patient in the at least one memory;
store at least one sequence-based treatment model in the at least one memory, wherein the at least one sequence-based treatment model is programmed to generate clinical report data as a function of sequence data;
generate patient-specific clinical report data in the at least one memory by executing the at least one sequence-based treatment model on at least one of the genome sequence, the exome sequence and the RNA sequence of the at least one patient, wherein the clinical report data comprises RNA transcription level for the genomic sequences;
generate a clinical report from the patient-specific clinical report data; and
cause an output device to present the clinical report, wherein the clinical report is annotated as
(a) false positive upon determination that the transcription level is below a threshold level or
(b) true positive upon determination that the transcription level is above the threshold level or
(c) false negative upon determination that the tumor and matched normal have no difference for a specific sequence, and where the transcription level is above the threshold level.

16. The system of claim 15 wherein the at least one multi-lane sequencing device is further configured to sequence at least eight patient's normal tissue and diseased in the common run, or wherein the at least one multi-lane sequencing device includes at least ten one multi-lane sequencing devices.

17. The system of claim 15 wherein the genome sequence comprises less than 20× reads, wherein the exome sequence comprises at least 150× reads, and wherein the RNA sequence comprises at least 150× reads.

18. The system of claim 15 wherein the exome sequence or the RNA sequence is enriched by at least a factor of 10 relative to the genome sequence.

19. The system of claim 15 wherein the at least one treatment model comprises an ensemble of treatment models, a trained treatment outcome prediction model, pathway expression model, a pathway recognition algorithm using data integration on genomic models (PARADIGM), or a drug response model.

20. The system of claim 15 wherein the at least one of the genome sequence, the exome sequence, and the RNA sequence is stored in the at least one memory according to a BAMBAM format.
